(19) United States
(12) Patent Application Publication
(10) Pub. No.: US 2026/0284388 A1
(43) Pub. Date: Sep. 24, 2026

(54) DEVICE FOR STRENGTHEN MUSCLES FOR PROSTATE AND URINARY INCONTINENCE

(71) Applicants: Kang Ho Song, Seoul (KR); GENERAL NET CORP., Seoul (KR)

(72) Inventor: Kang Ho Song, Seoul (KR)

(21) Appl. No.: 18/881,096

(22) PCT Filed: Jul. 14, 2023
(86) PCT No.: PCT/KR2023/010068

§ 371 Date: Jan. 3, 2025

(30) Foreign Application Priority Data
Jul. 14, 2022 (KR) 10-2022-0086730

(51) Int. Cl. A61N 1/36; A61N 1/04

(52) U.S. Cl. CPC A61N 1/36007; A61N 1/0452; A61N 1/0476; A61N 1/3603

(57) ABSTRACT

The present disclosure provides a muscle-strengthening stimulation electrode apparatus for the prostate and urinary incontinence, and more specifically, to a muscle-strengthening stimulation electrode apparatus designed to be used in contact with electrodes for the treatment of prostate conditions and urinary incontinence, where in the devices includes a lower plate placed on the chair, a housing coupled to the lower plate, an upper plate formed on the upper part of the housing; and electrodes placed on the upper plate.

### TECHNICAL FIELD

The present invention relates to a muscle-strengthening stimulation electrode apparatus for the prostate and urinary incontinence, and more specifically, to a muscle-strengthening stimulation electrode apparatus designed to be used in contact with electrodes for the treatment of prostate conditions and urinary incontinence.

### BACKGROUND ART

Urinary incontinence is a condition in which urine is involuntarily leaked, causing wetting of undergarments, which can lead to social and hygiene-related issues. This condition is very common in the urinary system, with approximately 40% of women in Korea experiencing urinary incontinence.

Prostate symptoms can be broadly categorized into three types. The first involves pain or discomfort in areas such as the perineum, around the anus, both inguinal regions, and the area above the pubic bone. The second relates to voiding symptoms associated with urination, such as weak urine flow, hesitancy, dribbling, and interrupted urine stream. The third pertains to storage symptoms within the bladder, including frequent urination, nocturia, urgency, and dysuria.

Various methods have been developed to strengthen the related muscles using low-frequency stimulation to improve urinary incontinence and prostate symptoms.

The prior art, such as Korean Patent No. 10-2201967, provides a medical composite device for treating urinary incontinence. However, in the prior technology, the electrode pad is symmetrically designed around the central axis. In reality, the lower body's shape when sitting is not symmetric along the upper and lower parts centered on the pelvic region. Designs that fail to consider the human body's shape cannot provide appropriate electrical stimulation to specific body parts, thereby reducing therapeutic efficiency.

Moreover, the existing design lacks guiding mechanisms to ensure proper sitting posture during use.

### DISCLOSURE OF THE INVENTION

### Technical Problem

The objective of one embodiment of the present invention is to provide a therapeutic device equipped with electrodes designed to conform to the human body shape, thereby enhancing treatment efficiency.

Additionally, another objective of this embodiment is to offer a therapeutic device with an ergonomic design that minimizes discomfort and fatigue by considering the curvature of the user's legs, while ensuring an appropriate degree of leg separation for proper posture.

Moreover, this embodiment aims to provide a therapeutic device where the electrode units adhere evenly to the user's body, thereby increasing the effectiveness of the treatment.

Furthermore, this embodiment seeks to deliver a therapeutic device equipped with a preheating feature, ensuring the user is not discomforted by low temperatures during cold weather conditions, such as in winter.

The challenges addressed by the present invention are not limited to those mentioned above, and other objectives not explicitly stated here can be clearly understood by those skilled in the art through the description below.

### Technical Solution

An embodiment of the present invention, designed to address the aforementioned technical challenges, involves a muscle-strengthening stimulation electrode apparatus for the prostate and urinary incontinence. This apparatus is mounted on a chair, covered with a wet tissue, and used while the user is seated on it. The device comprises s lower plate mounted on the chair, a housing coupled to the lower plate, an upper plate formed on the housing, and electrodes arranged on the upper plate. The electrodes include: a central first electrode positioned symmetrically along the left and right sides, second anterior electrodes and second posterior electrodes symmetrically arranged around the first electrode, and third anterior electrodes and third posterior electrodes symmetrically arranged around the second electrodes.

The second anterior electrodes are formed with angular edges, while the second posterior electrodes have rounded edges.

The electrodes protrude from the surface of the upper plate and are spaced apart to create a spatial area. The protrusion height of the electrodes and the spacing between them form this spatial area, which prevents the wet tissue from coming into contact with the surface of the upper plate. This configuration ensures efficient and effective use of the device.

### ADVANTAGEOUS EFFECTS

An embodiment of the present invention provides electrodes tailored to the human body shape, resulting in enhanced treatment efficiency.

Additionally, this embodiment includes a leg guide at the front center, designed with a more ergonomic shape that considers the curvature of the user's legs. This minimizes discomfort, eliminates fatigue, and ensures an appropriate degree of leg separation for optimal posture.

Moreover, this embodiment incorporates elastic materials within the electrode unit and provides space at the bottom of the electrode unit for vertical movement. This allows the electrode unit to adhere evenly to the user's body through elasticity, thereby enhancing the therapeutic effect.

Furthermore, the invention features a temperature control unit, enabling preheating to a suitable temperature to prevent discomfort from cold temperatures, especially during winter.

The benefits of the present invention are not limited to those mentioned above. Additional unaddressed challenges and advantages can be clearly understood by those skilled in the art through the descriptions provided below.

### BRIEF DESCRIPTION OF THE DRAWINGS

FIG. 1 is a perspective view of an embodiment of the present invention.

FIG. 2 is a top plan view of an embodiment of the present invention.

FIG. 3 is a front view of an embodiment of the present invention.

FIG. 4 is a view illustrating the usage state of an embodiment of the present invention.

FIG. 5 is another view illustrating the usage state of an embodiment of the present invention.

FIG. 6 is an enlarged view of the “upper plate” and “electrodes” shown in FIG. 1.

FIG. 7 is a cross-sectional view illustrating the usage state of an embodiment of the present invention.

FIG. 8 is another view illustrating the usage state of an embodiment of the present invention.

FIG. 9 is a conceptual diagram illustrating human pelvic muscles.

FIG. 10 is another conceptual diagram illustrating human pelvic muscles.

FIG. 11 is a view illustrating the usage state of an embodiment of the present invention.

FIG. 12 is a conceptual diagram showing the principle of muscle contraction induced by electrical stimulation.

FIG. 13 is a schematic diagram illustrating the effects of the muscle-strengthening stimulation electrode apparatus for the prostate and urinary incontinence, according to the present invention.

FIG. 14 is another schematic diagram illustrating the effects of the muscle-strengthening stimulation electrode apparatus for the prostate and urinary incontinence, according to the present invention.

FIG. 15 is yet another schematic diagram illustrating the effects of the muscle-strengthening stimulation electrode apparatus for the prostate and urinary incontinence, according to the present invention.

### BEST MODE FOR CARRYING OUT THE INVENTION

Hereinafter, the embodiments of the present invention will be described in more detail with reference to the accompanying drawings. However, the present invention is not limited to the embodiments disclosed below but may be implemented in various other forms. The embodiments are provided to make the disclosure of the present invention complete and to fully inform those skilled in the art of the scope of the invention. The same reference numerals in the drawings refer to the same elements.

When adding reference numerals to the components of each drawing, it should be noted that the same components are given the same numerals as much as possible, even if they are shown in different drawings. Also, in describing the present invention, detailed descriptions of related known configurations or functions are omitted if they are deemed to obscure the gist of the present invention.

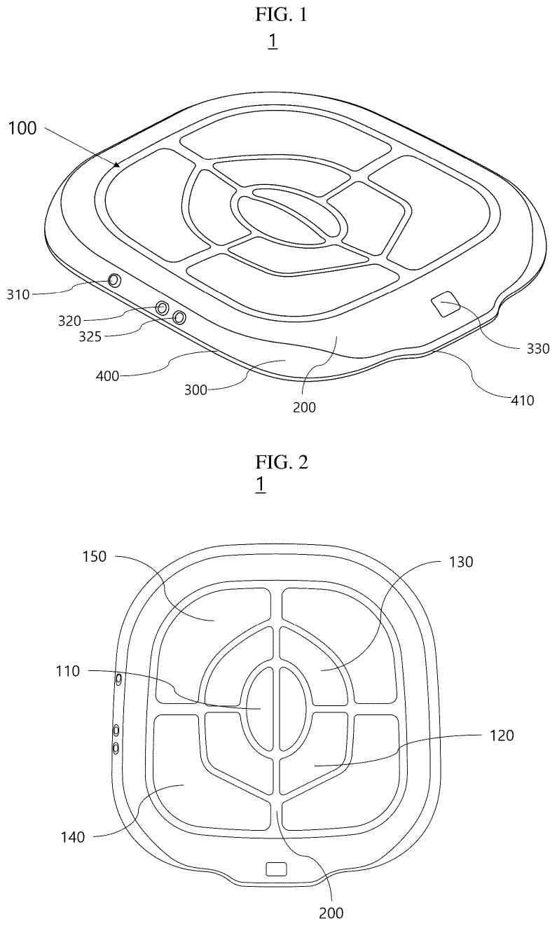

FIG. 1 is a perspective view of an embodiment of the present invention.

FIG. 2 is a plan view of an embodiment of the present invention.

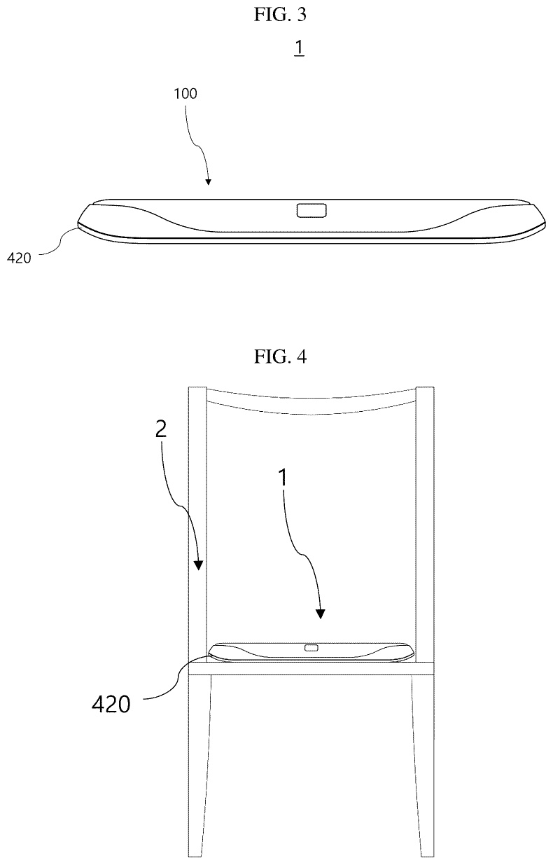

FIG. 3 is a front view of an embodiment of the present invention.

FIG. 4 is a usage state diagram of an embodiment of the present invention.

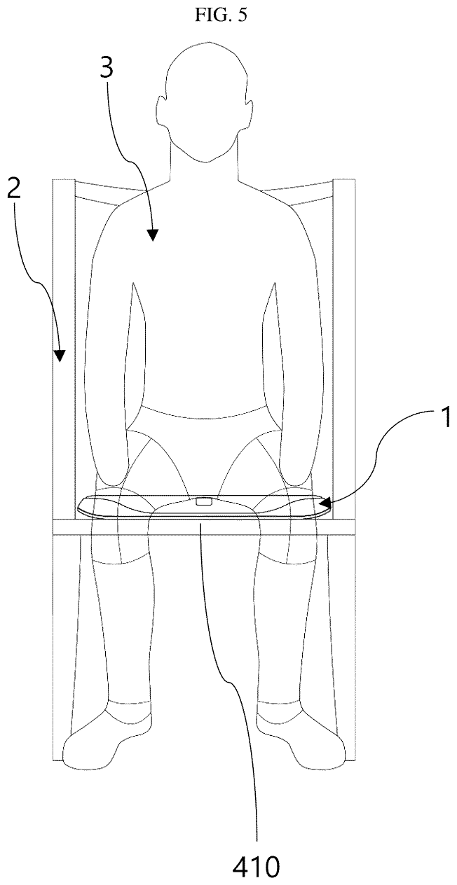

FIG. 5 is another usage state diagram of an embodiment of the present invention.

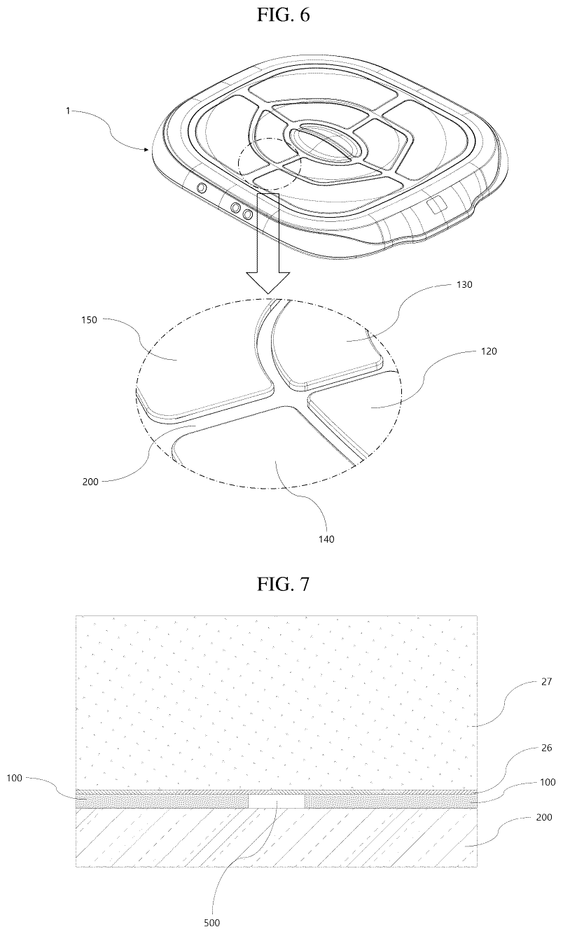

FIG. 6 is an enlarged view of the “upper plate (200)” and “electrode (100)” in FIG. 1.

FIG. 7 is a cross-sectional view for explaining the usage state of an embodiment of the present invention.

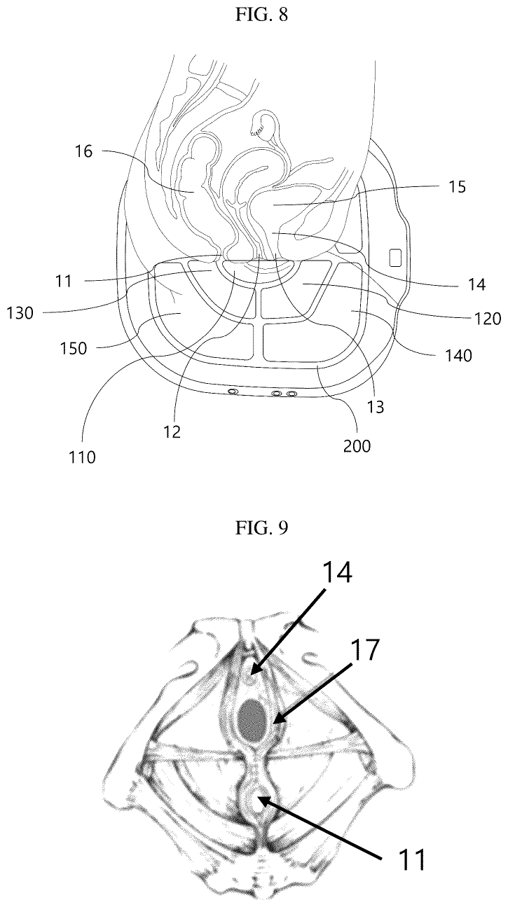

FIG. 8 is another usage state diagram of an embodiment of the present invention.

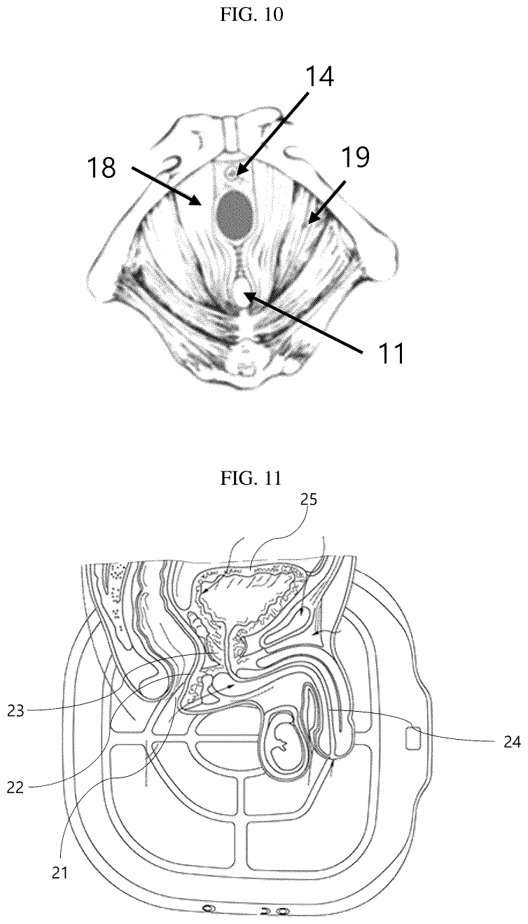

FIG. 9 is a conceptual diagram showing the pelvic muscles of the human body.

FIG. 10 is another conceptual diagram showing the pelvic muscles of the human body.

FIG. 11 is another usage state diagram of an embodiment of the present invention.

FIG. 12 is a conceptual diagram showing the principle of muscle contraction in the human body by electrical stimulation.

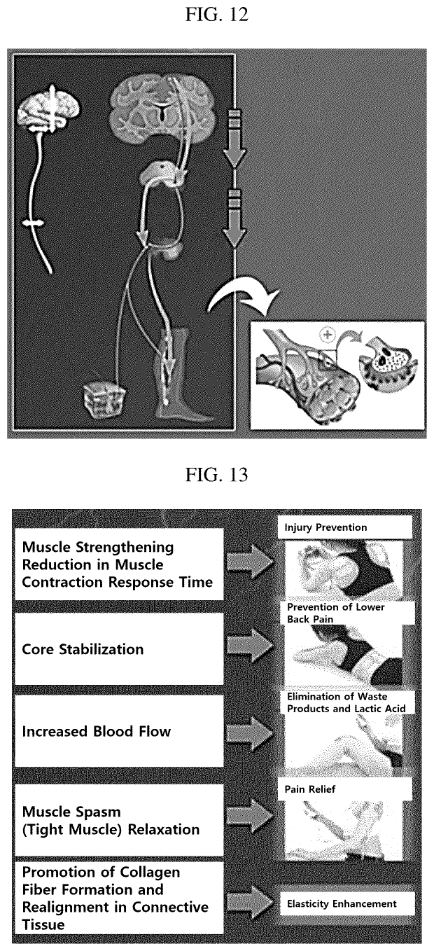

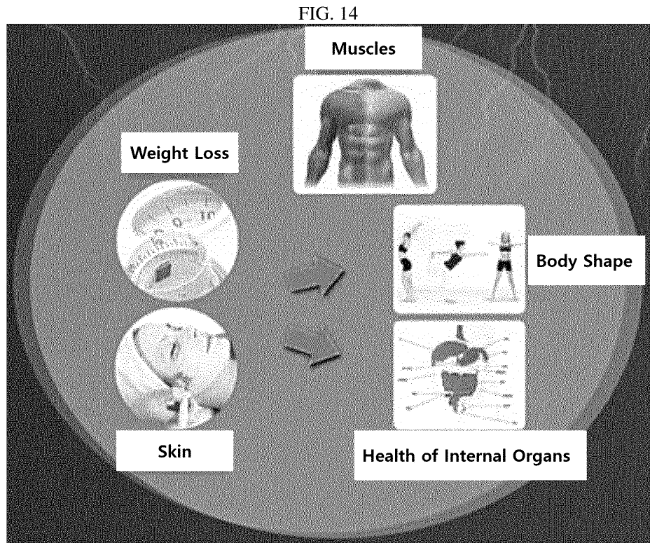

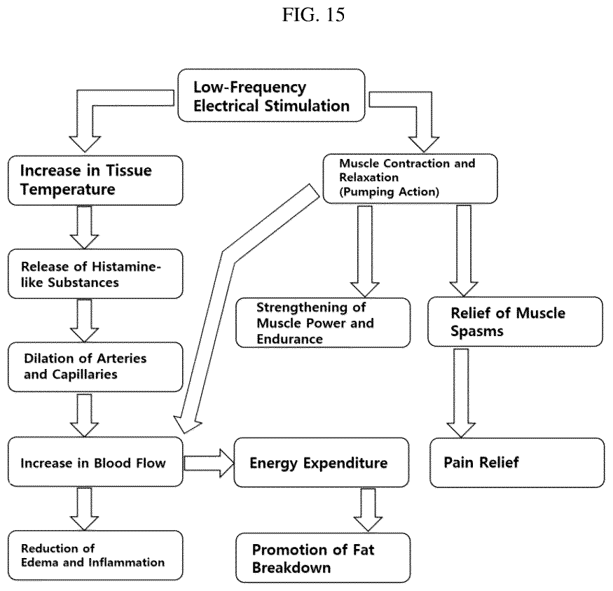

FIGS. 13 to 15 are schematic diagrams showing the effects of the muscle strengthening stimulation electrode device (1) for prostate and urinary incontinence according to the present invention.

An embodiment of the present invention relates to a muscle strengthening stimulation electrode device 1 for prostate and urinary incontinence. This device is placed on a chair 2 and covered with a wet tissue 26, on which the user 3 sits. As shown in FIGS. 1 to 3 and 6 to 7, it includes electrodes 100, an upper plate 200, a housing 300, a lower plate 400, and a space part 500.

The lower plate 400 is the part that is placed on the chair. It may have a leg guide 410 protruding forward, which guides the legs to spread naturally, ensuring the pelvis is in close contact with the electrodes 100 for effective electrical stimulation. The leg guide 410 is designed to guide the legs to an appropriate width, as shown in FIG. 5, ensuring the pelvis is in close contact with the electrodes 100 for effective electrical stimulation.

Additionally, the lower plate 400 may have an inclined grip part 420 on its side, forming a groove between the upper plate (200 and the lower plate 400 when placed on the chair, allowing the user 3 to easily carry the device by hand.

The housing 300 is the part that is coupled to the lower plate 400. It may include electrical components, electronic circuits, and control units, as well as operation parts 310, 320, 325, and 330 for operating the device.

The upper plate 200 is formed on the upper part of the housing. It is made of an insulator.

The electrodes 100 are placed on the upper plate 200 and include more than two electrodes 100 to generate a current between them, stimulating the user's 3 muscles. The electrodes 100 include a first electrode 110 placed centrally, a second front electrode 120 and a second rear electrode 130 symmetrically surrounding the first electrode 110, and a third front electrode 140 and a third rear electrode 150 symmetrically surrounding the second front electrode 120 and the second rear electrode 130. The electrodes 100 are formed to protrude from the upper plate 200 and are spaced apart to form a space part 500, ensuring the wet tissue 26 does not contact the surface of the upper plate 200.

Referring to FIGS. 5 and 6, the user 3 covers the electrodes 100 with a conductive wet tissue 26 and sits on it to use the muscle strengthening stimulation electrode device 1 for prostate and urinary incontinence. The electrodes 100 pass a low-frequency current, and the wet tissue 26 transmits this current to the user's 3 body. A space part 500 is formed between the spaced-apart electrodes 100. In this embodiment, the space part 500 ensures that the wet tissue 26 does not contact the surface of the upper plate 200 due to the spacing and protrusion height of the electrodes 100.

When the wet tissue contacts the upper plate, the low-frequency current generated by the electrodes and the wet tissue is attenuated by the upper plate, reducing the low-frequency current delivered to the human body. This embodiment of the invention prevents the wet tissue from contacting the upper plate, thereby preventing the attenuation of the low-frequency current.

The second front electrode 120 is characterized by having an angular outer edge, while the second rear electrode 130 has a rounded outer edge. Similarly, the third front electrode 140 has an angular outer edge, and the third rear electrode 150 can have a rounded outer edge. The angular outer edges of the second front electrode 120 and the third front electrode 140 are designed to correspond to the anatomical structure of the human body. In particular, the angular outer edge of the second front electrode 120 is shaped to effectively provide electrical stimulation to major pelvic muscles such as the pubococcygeus and iliococcygeus muscles.

FIGS. 8, 9, and 10 illustrate the relationship between the electrodes 100 and the body, and the important pelvic muscles targeted for treatment in this embodiment of the invention. In this embodiment, the bulbospongiosus muscle 17, which is the innermost muscle, is primarily stimulated by the first electrode 110. The puborectalis muscle 18 is stimulated by the second front electrode 120 and the second rear electrode 130, while the pubococcygeus muscle 19 is stimulated by the third front electrode 140 and the third rear electrode 150. However, the range of stimulation may vary depending on the size and shape of the individual's body.

For males, as shown in FIG. 11, the prostate 23 can be stimulated for treatment using the first electrode 110, the second front electrode 120, and the second rear electrode 130. The rounded outer edges of the second rear electrode 130 and the third rear electrode 150 are also designed to correspond to the anatomical structure of the human body. In particular, the rounded outer edge of the second front electrode 120 is shaped to effectively provide electrical stimulation to major pelvic muscles such as the pubococcygeus muscle.

Additionally, the electrodes 100 may include an elastic material inside, allowing them to be elastic vertically. This elasticity ensures that the electrodes 100 adhere evenly to the user's 3 pelvis, allowing the electrical stimulation to be delivered more effectively.

The muscle strengthening stimulation electrode device 1 for prostate and urinary incontinence may also include a temperature control unit. The temperature control unit preheats the upper plate 200, ensuring that the user 3 does not feel cold even during winter.

The principle of muscle contraction in the user 3 who receives electrical stimulation from the muscle strengthening stimulation electrode device 1 for prostate and urinary incontinence is illustrated in FIG. 12. The effects of muscle contraction due to electrical stimulation are shown in FIGS. 13 to 15.

The above description is merely illustrative of the technical concept of the present invention, and various modifications and changes can be made without departing from the essential characteristics of the present invention by those skilled in the art to which the present invention pertains.

Therefore, the embodiments disclosed in the present invention are not intended to limit the technical concept of the present invention but to explain it. The scope of the technical concept of the present invention is not limited by these embodiments. The protection scope of the present invention should be interpreted according to the following claims, and all technical concepts within the equivalent scope should be interpreted as being included in the scope of rights of the present invention.

1. A prostate and urinary incontinence electrode device, which is placed on a chair, covered with a wet tissue, and used by a user sitting on it, comprising:
a lower plate placed on the chair;
a housing coupled to the lower plate;
an upper plate formed on the upper part of the housing; and
electrodes placed on the upper plate,
wherein the electrodes include a first electrode placed centrally, a second front electrode and a second rear electrode symmetrically surrounding the first electrode, and a third front electrode and a third rear electrode symmetrically surrounding the second front electrode and the second rear electrode,
wherein the electrodes are formed to protrude from the upper plate and are spaced apart to form a space part, preventing the wet tissue from contacting the surface of the upper plate to avoid attenuation of the low-frequency current,
wherein a leg guide protruding forward is formed on the lower plate, guiding the legs to spread naturally, ensuring the pelvis is in close contact with the electrodes,
wherein the second front electrode has an angular outer edge corresponding to the anatomical structure of the human body, and the second rear electrode has a rounded outer edge,
wherein the third front electrode has an angular outer edge corresponding to the anatomical structure of the human body, and the third rear electrode has a rounded outer edge.

2. The prostate and urinary incontinence electrode device of claim 1, wherein an inclined grip part is formed on the side of the lower plate.

3. The prostate and urinary incontinence electrode device of claim 1, further comprising a temperature control unit.

4. The prostate and urinary incontinence electrode device of claim 1, wherein the electrodes include an elastic material inside, allowing them to be elastic vertically.

5. A prostate and urinary incontinence electrode device, which is placed on a chair, covered with a wet tissue, and used by a user sitting on it, comprising:
a lower plate placed on the chair;
a housing coupled to the lower plate;
an upper plate formed on the upper part of the housing;
electrodes placed on the upper plate; and
a temperature control unit,
wherein the electrodes include a first electrode placed centrally, a second front electrode and a second rear electrode symmetrically surrounding the first electrode, and a third front electrode and a third rear electrode symmetrically surrounding the second front electrode and the second rear electrode.

6. The prostate and urinary incontinence electrode device of claim 5, wherein an inclined grip part is formed on the side of the lower plate.

7. The prostate and urinary incontinence electrode device of claim 5, wherein the electrodes include an elastic material inside, allowing them to be elastic vertically.
